(19) United States
(12) Patent Application Publication
(10) Pub. No.: US 2011/0288598 A1
(43) Pub. Date: Nov. 24, 2011

(54) LOCKING SCREW DEVICE

(75) Inventors: Berton R. Moed, St. Louis, MO (US); Bernard L. Randall, Macomb, MI (US); Christopher John Rudy, Fort Gratiot, MI (US)

(73) Assignee: SAINT LOUIS UNIVERSITY, St. Louis, MO (US)

(21) Appl. No.: 13/061,515

(22) PCT Filed: Sep. 4, 2009
(86) PCT No.: PCT/US2009/004985

§ 371 Date: May 24, 2011

Related U.S. Application Data
(60) Provisional application No. 61/191,343, filed on Sep. 8, 2008.

(51) Int. Cl. A61B 17/86

(52) U.S. Cl. 606/303

(57) ABSTRACT

Locking screw system includes screw and female threaded receiver that provide for and lock through interference fitting, which may be from major-major and/or minor-minor diameter interference. The system may be embodied as an orthopedic implant or implant component.

This claims priority beneits of United States of America (U.S.) provisional patent application No. U.S. 61/191,343 filed on Sep. 8, 2008 A.D. For the US, the same is claimed under the Patent Cooperation Treaty and/or Title 35 United States Code, notably under sections 119(e), 120, 363 and/or 365. Where applicable, as in the US, the specification of that application in its entirety, which of course includes its drawings, is incorporated herein by reference.

### FIELD AND PURVIEW OF THE INVENTION

In one aspect, this concerns and discloses an implant system, which includes a screw that locks to another part of the system through an interference fit. In another aspect, this concerns and discloses a screw system that includes a male threaded screw and a female threaded receiver, as an example, a nut, that lock through major-major and/or minor-minor diameter interference, which may include employment in an implant or other mechanical system.

### BACKGROUND TO THE INVENTION

A problem in the art is instability of some long bone plate or nail ensembles fastened to the bone by screws. One or more of the screws may loosen in the bone and cause the instability. For example, various femoral and tibial nail ensembles employ screws that are threaded through cortical bone, into threaded holes in the nail with a bushing that is placed in a reamed out medullary canal, and, passing through the nail, into an opposing portion of cortical bone. Such ensembles may be touted as being locking. Yet, the problem of instability and loosening remains with such ensembles.

Randall et al., publication No. US 2002/0087161 A1 and patent No. U.S. Pat. No. 6,635,059 B2, discloses a cannulated locking screw system especially for transiliac implant. Thereby, major-minor, pitch-diameter, locking interference fitting can be provided. Compare, Moed et al., J. TRAUMA Injury, Vol. 62, No. 2, pages 357-364 (February 2007).

The problem of loosening also can occur in classic mechanical situations. The SPIRALOCK tap of H.D. Holmes/Detroit Tool Industries is a patented female self-locking thread form based on a simple wedge lock principle, which is intended to address such situations.

It would be desirable to ameliorate if not solve one or more of the problems in the art. It would be desirable to provide one alternative or more to the art.

### A FULL DISCLOSURE OF THE INVENTION

The present invention provides an implant system, which includes a screw that locks to another part of the system through an interference fit, excepting that the system is not that of the aforementioned publication and patent to Randall et al. Although such an implant system can have major-minor, pitch-diameter, locking interference fitting, it can have major-major and/or minor-minor diameter interference provided with a screw system that includes a male threaded screw and a female threaded receiver, examples of which include a nut, a threaded plate or other member, that lock through major-major and/or minor-minor diameter interference, which may include employment in an implant or other mechanical system. A kit including the screw and the other part or female threaded receiver is also provided.

The invention is useful in surgical repair of a patient. It is also useful in numerous other mechanical applications.

Significantly, by the invention, the art is advanced in kind. In particular embodiments, long bone plate or nail systems can be fastened to the bone by screws with an interference fit, thus stabilizing the system with particular respect to the screw and the plate or nail such that the system is intrinsically stable and does not necessarily or primarily rely on screw to bone union for stability. The interference fit itself can be very effective. And so, long bone repair can be more effective. Also, for instance, total joint replacement parts such as for the knee, hip and shoulder can be screwed in with more stability. The present implant system can be applied with respect to any suitable arthroplasty surface where an implant component is screwed to an underlying substrate, especially to a surface of bone, whether altered such as by resection, milling, etc., or not so altered. Further, the invention, with its major-major and/or minor-minor diameter interference can be applied in numerous other mechanical situations, especially where vibration and/or motion may otherwise undesirably loosen a screwed-in fastening, for example, applications in aircraft, automotive, bridges, buildings, cabinetry, dies and presses, electrical, electro-mechanical, marine vessels, pipeline connection, pumps, weaponry, and so forth and the like, to provide effective locking interference for secure fastening. Control of torque to an outstanding degree can be provided. Also, a follow up chance may be afforded with major-major and/or minor-minor diameter interference should a preceding screw in the major-major system or preceding female threaded receiver in the minor-minor system have a body of a less than desirable size, for example, length, or configuration. It is relatively easy and efficient to make and use the major-major and minor-minor interference system.

Numerous further advantages attend the invention.

The drawings form part of the specification hereof. With respect to the drawings, which are not necessarily drawn to scale, the following is briefly noted:

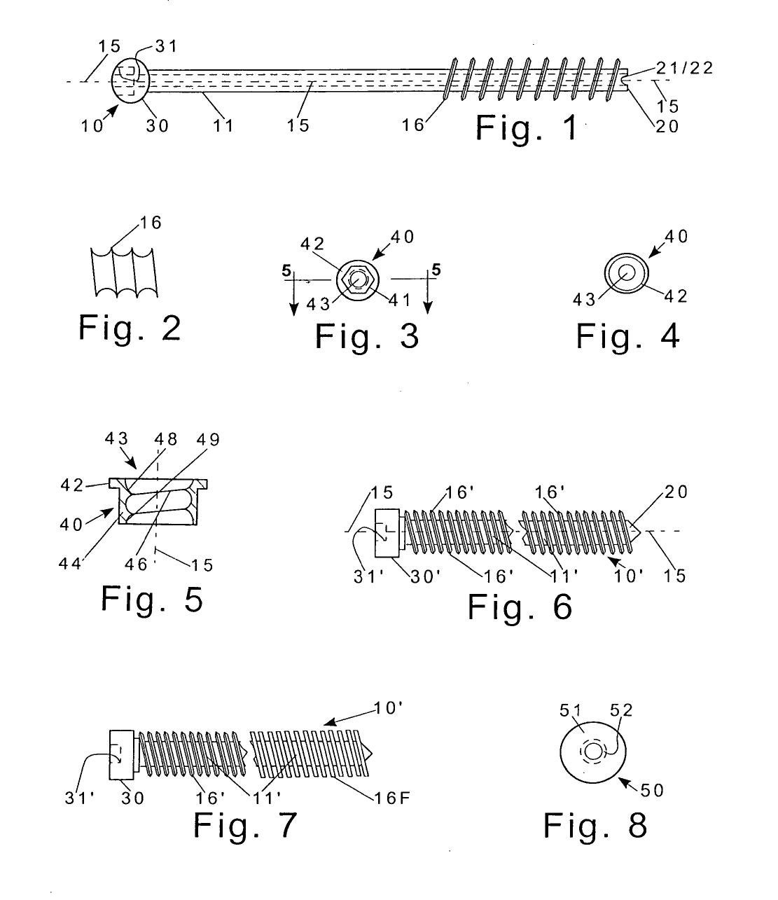

FIG. 1 is a side plan view of a cannulated screw that may be employed in the practice of the present invention.

FIG. 2 is a side view of screw threads.

FIG. 3 is a top view of a nut that may be employed in the practice of the present invention.

FIG. 4 is a bottom view of the nut of FIG. 3.

FIG. 5 is a sectional view of the nut of FIG. 3, taken along 5-5 in FIG. 3.

FIG. 6 is a side plan view of a non-cannulated screw that may be employed in the practice of the present invention.

FIG. 7 is a side plan view of the screw of FIG. 6 showing deformation of some oversized threads as by flattening after passing through an orifice.

FIG. 8 is a top view of a washer that may be employed in the practice of the present invention.

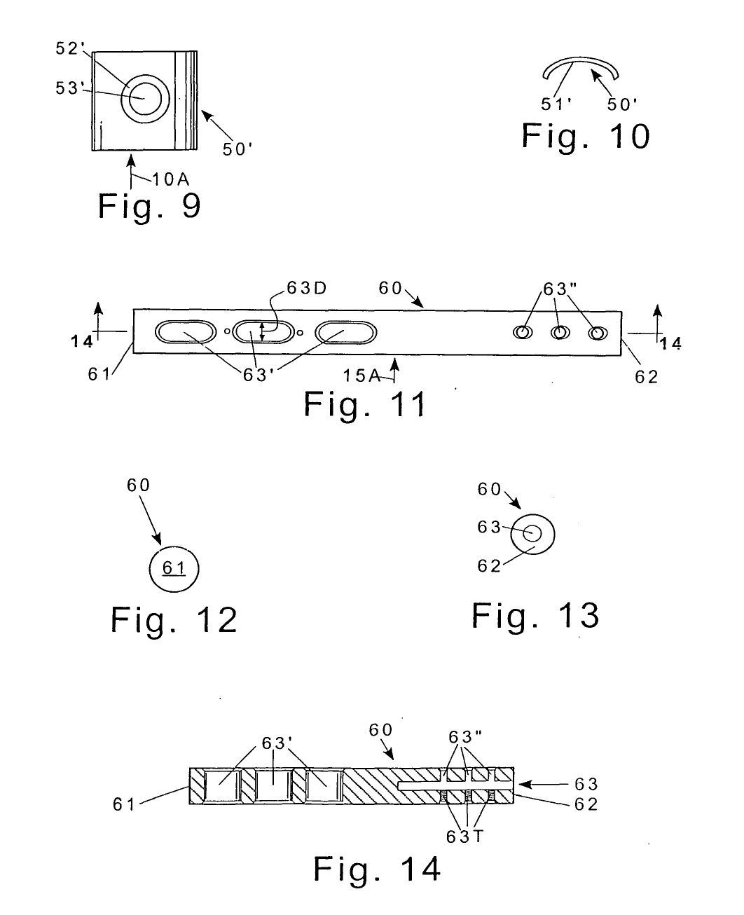

FIG. 9 is a top view of another washer that may be employed in the practice of the present invention.

FIG. 10 is a side view of the washer of FIG. 9, taken along the direction of arrow 10A in FIG. 9.

FIG. 11 is a top view of a long bone nail that may be employed in the practice of the present invention, for example, as a humeral or similarly sized long bone implant part.

FIG. 12 is a first end view of the nail of FIG. 11.

FIG. 13 is a second end view of the nail of FIG. 11.

FIG. 14 is a sectional view of the nail of FIG. 11, taken along 14-14 in FIG. 11.

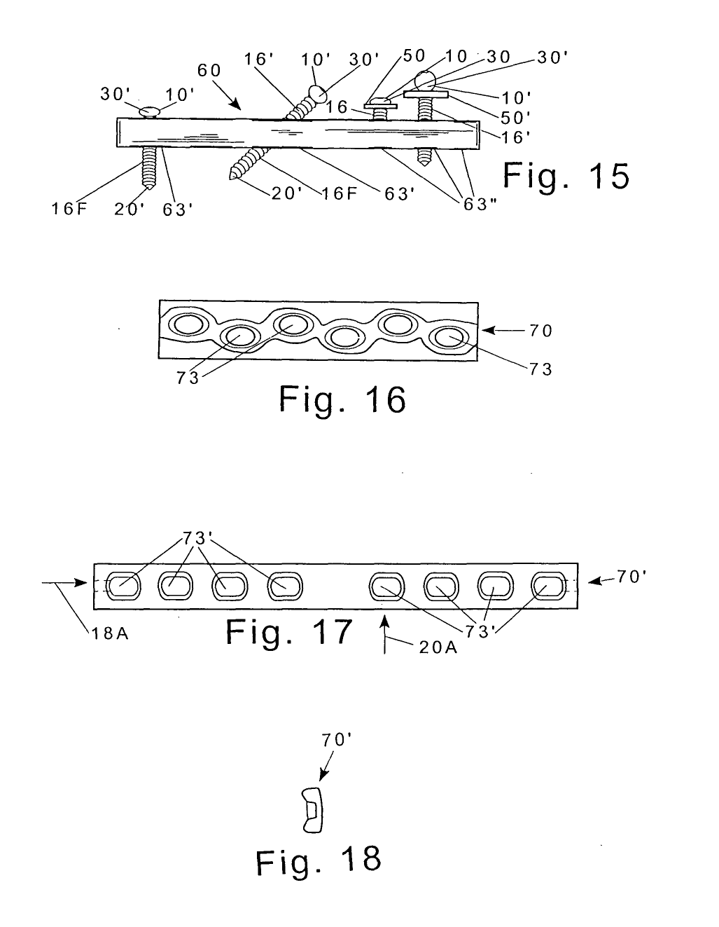

FIG. 15 is a side view of the nail of FIG. 11, taken along the direction of arrow 15A in FIG. 11, with screws as of FIGS. 1 and 6 in place in interference fittings with the nail.

FIG. 16 is a top view of a long bone plate that may be employed in the practice of the present invention.

FIG. 17 is a top view of another long bone plate that may be employed in the practice of the present invention.

FIG. 18 is an end view of the plate of FIG. 17, taken along the direction of arrow 18A in FIG. 17.

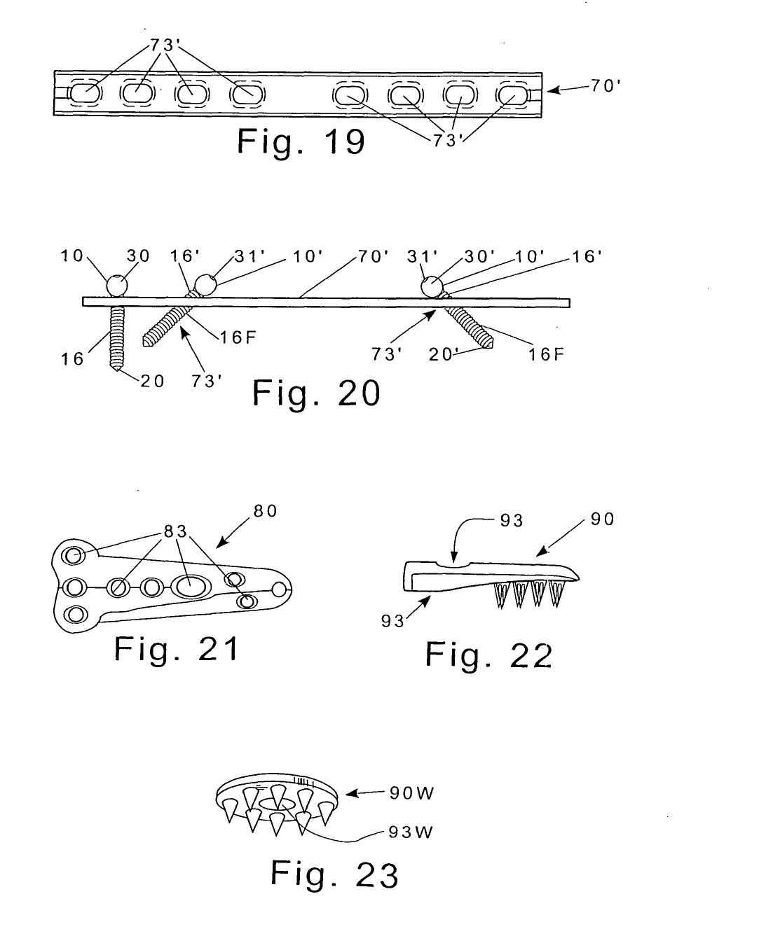

FIG. 19 is a bottom view of the plate of FIG. 17.

FIG. 20 is a side view of the plate of FIG. 17, taken along the direction of arrow 20A in FIG. 17, with screws as of FIGS. 1 and 6 in place in interference fittings with the plate.

FIG. 21 is a top view of another plate, a spoon plate, that may be employed in the practice of the present invention.

FIG. 22 is a side view of a ligament fixation plate that may be employed in the practice of the present invention.

FIG. 23 is a bottom, perspective view of a ligament fixation washer that may be employed in the practice of the present invention.

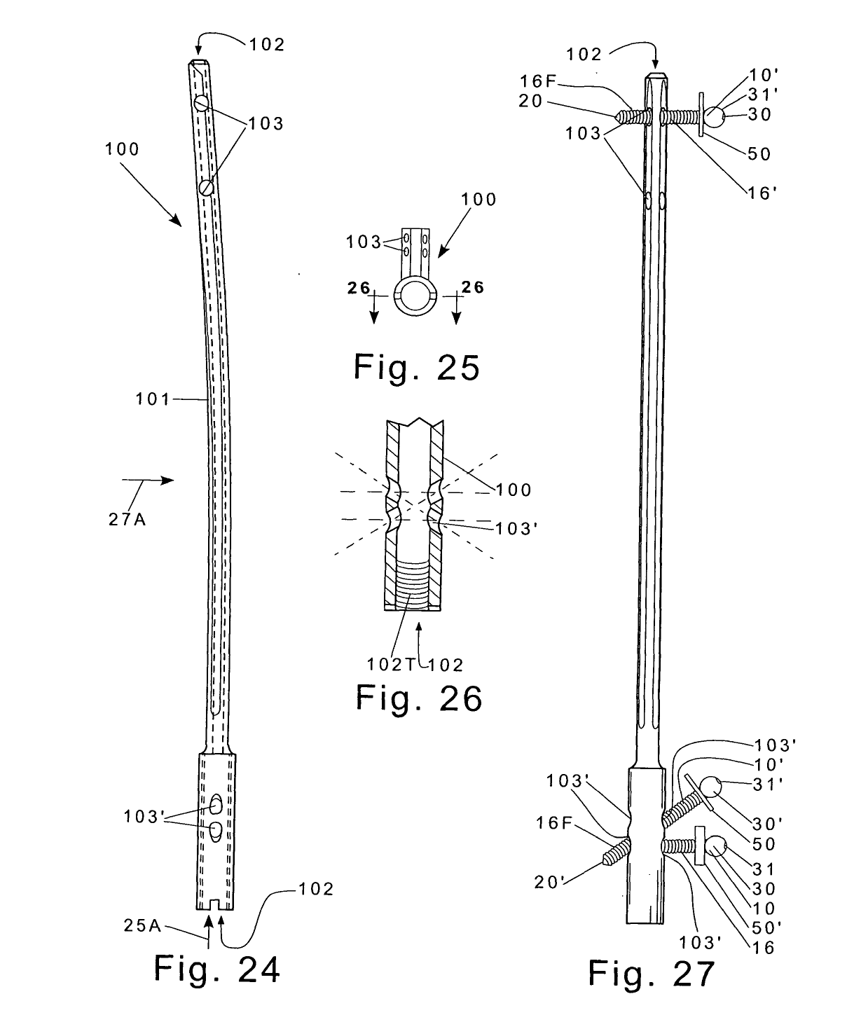

FIG. 24 is a side plan view of a femoral nail that may be employed in the practice of the present invention.

FIG. 25 is an end view of the nail of FIG. 24, taken along the direction of arrow 25A in FIG. 24.

FIG. 26 is a sectional view of part of the end of the nail of FIG. 24, taken along 26-26 in FIG. 25.

FIG. 27 is a side view of the nail of FIG. 24, taken along the direction of arrow 27A in FIG. 24, with screws as of FIGS. 1 and 6 in place in interference fittings with the nail.

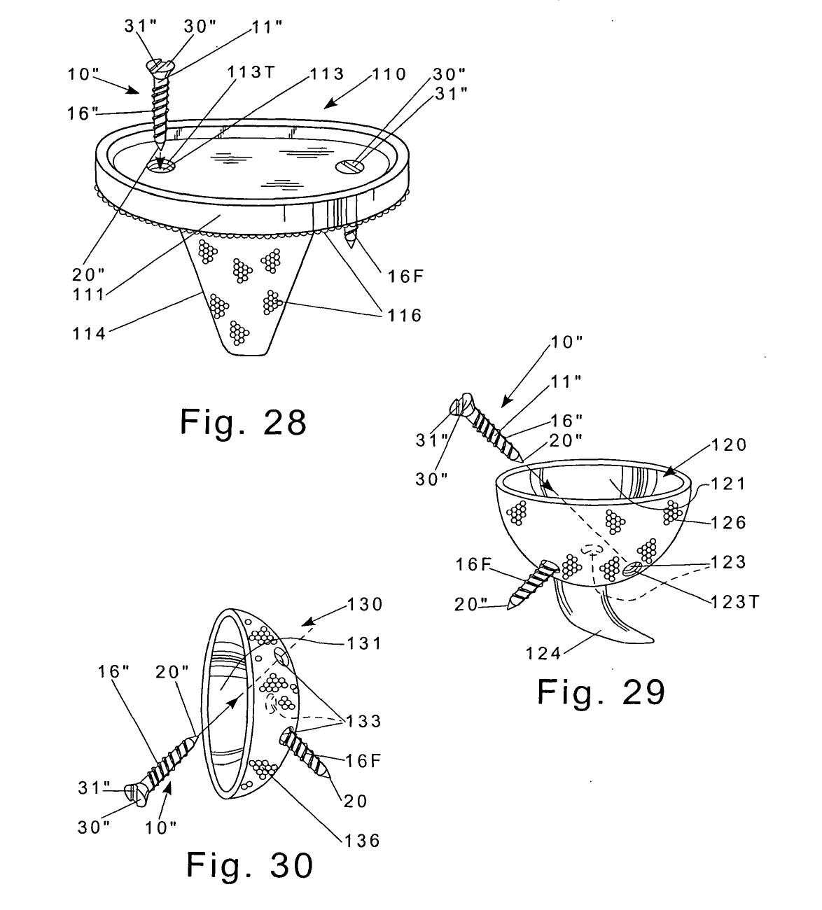

FIG. 28 is a perspective plan view of a tibial tray sans liner for a total knee replacement joint, and flat top screws, which may be employed in the practice of the present invention.

FIG. 29 is a perspective plan view of an acetabular shell sans cup for a total hip replacement joint, and flat top screws, which may be employed in the practice of the present invention.

FIG. 30 is a perspective plan view of an glenoid shell sans cup for a total shoulder replacement joint, and flat top screws, which may be employed in the practice of the present invention.

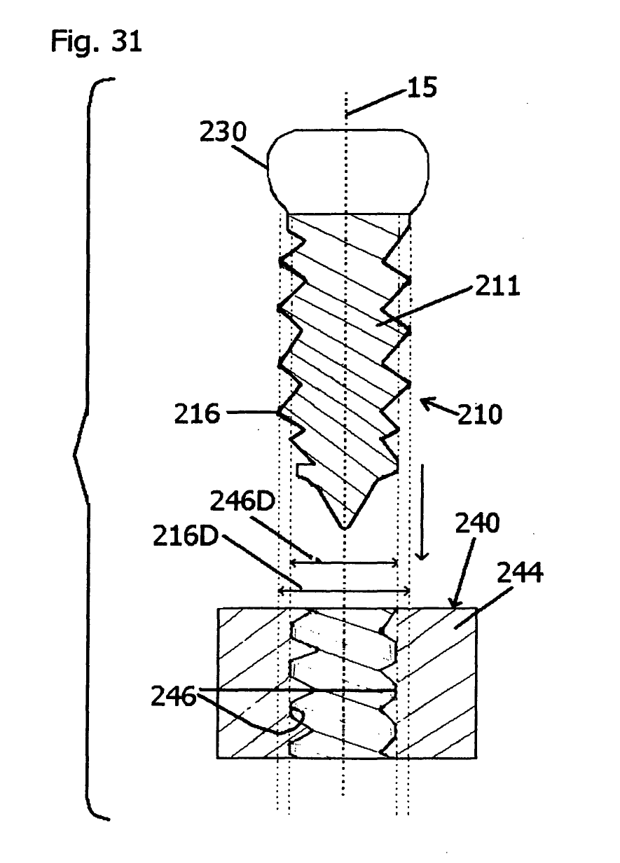

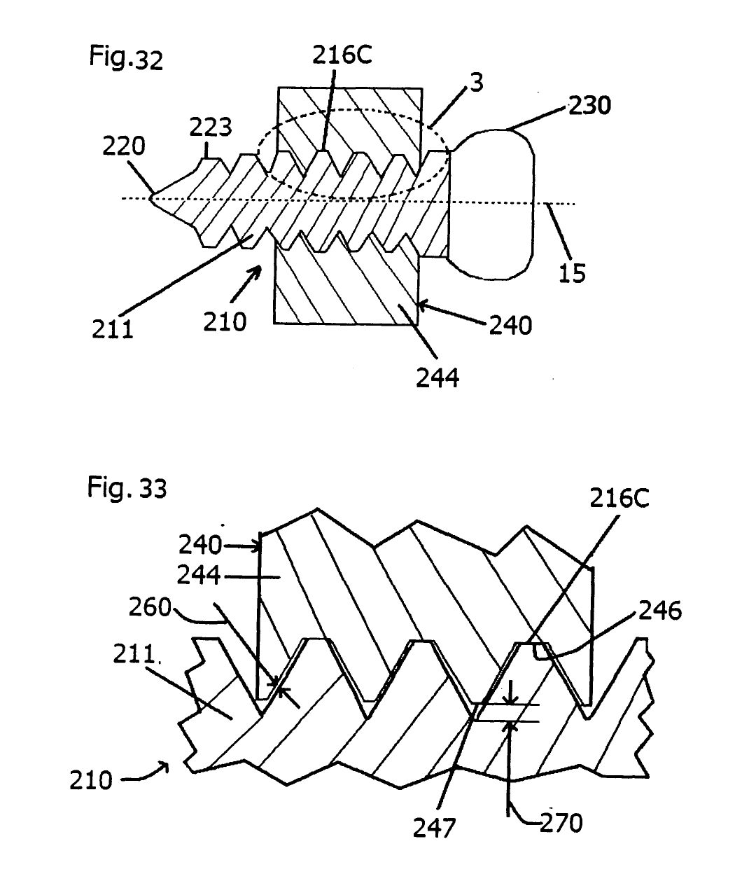

FIG. 31 is an exploded, side plan view in part section of a locking screw system that employs major-major diameter interference, unassembled. This embodiment can be employed as part of an orthopedic implant.

FIG. 32 is a side plan view in part section of the locking screw system of FIG. 31, assembled.

FIG. 33 is a detailed view of part of the locking screw system of FIG. 31, taken from within circle 3 of FIG. 32.

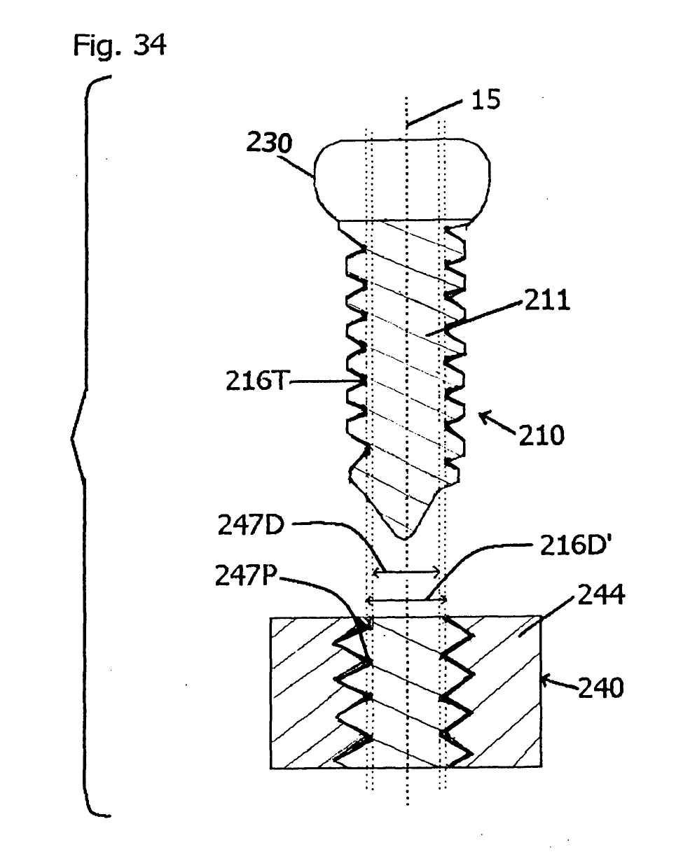

FIG. 34 is an exploded, side plan view in part section of a locking screw system that employs minor-minor diameter interference, unassembled. This embodiment can be employed as part of an orthopedic implant.

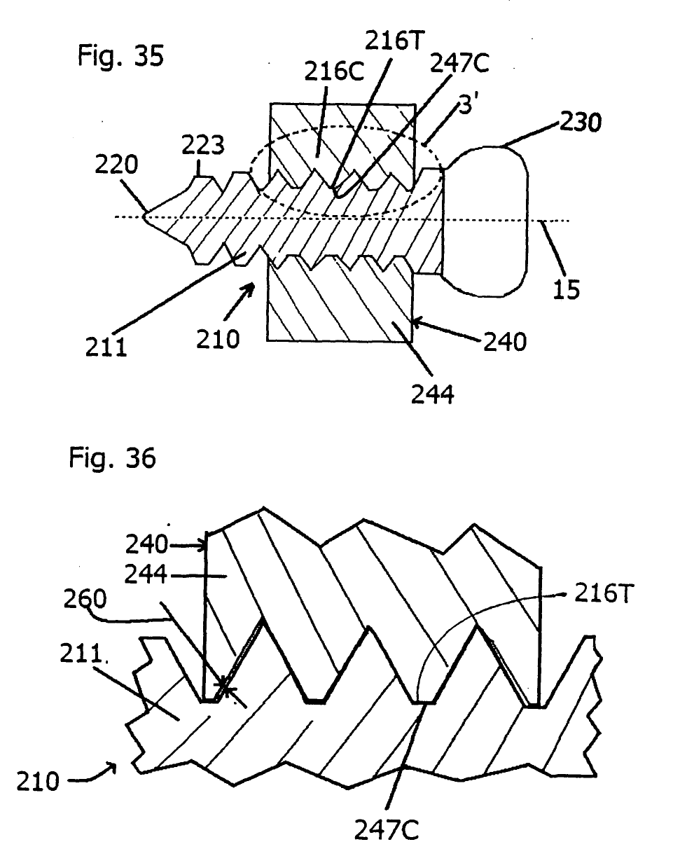

FIG. 35 is a side, plan view in part section of the locking screw system of FIG. 34, assembled.

FIG. 36 is a detailed view of part of the locking screw system of FIG. 34, taken from within circle 3′ of FIG. 35.

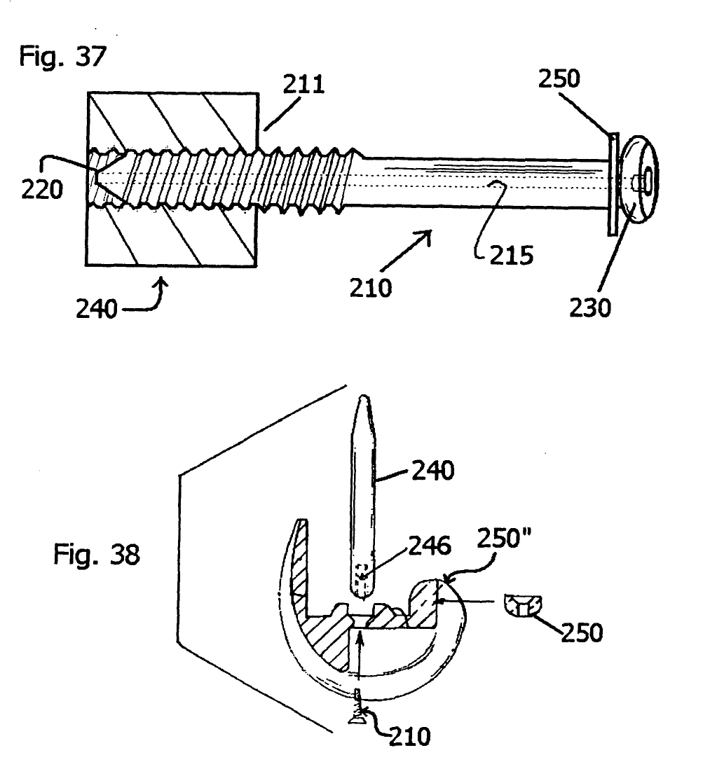

FIG. 37 is a side plan view in part section of a locking screw system having major-major and minor-minor diameter interference. The screw is cannulated; a washer is present.

FIG. 38 is an exploded side plan view in part section of a locking screw system having major-major and/or minor-minor diameter interference. It is embodied as a knee implant femoral component having a spike that acts as a female threaded receiver, a first washer, a condyle-containing body that may be considered to correspond to a second washer, and the screw.

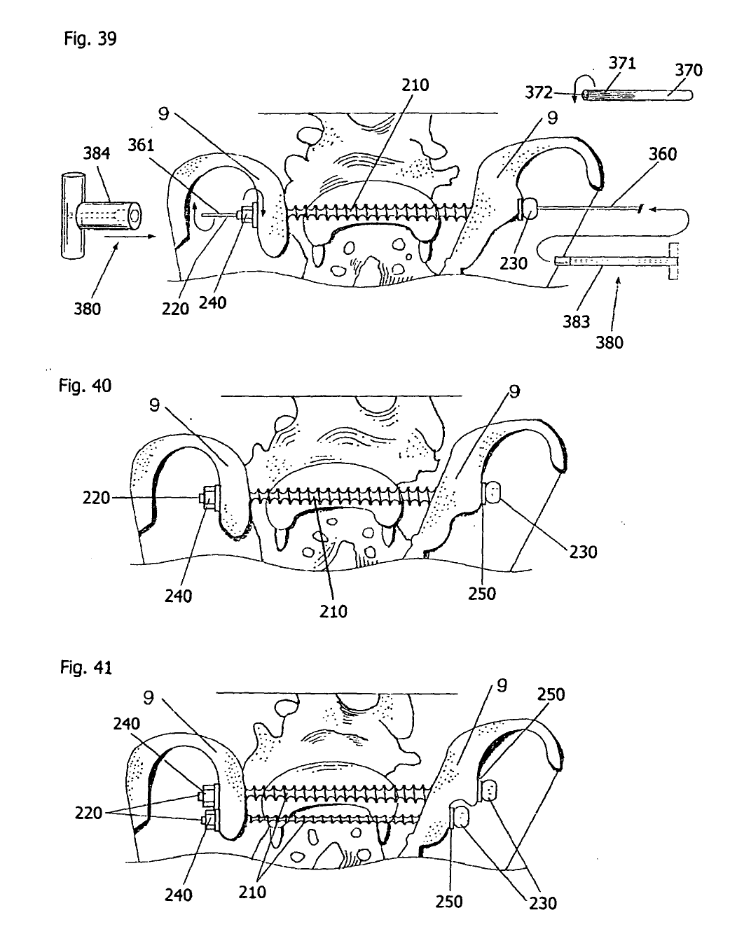

FIG. 39 is a rear view of a locking screw system embodied as a cannulated screw especially for transiliac implant, which has major-major and/or minor-minor diameter interference, and which is implanted in a human pelvic girdle so as to stabilize the sacrum, with the assistance of a cannulated starter drill, a guidewire, and a set of two cannulated tools.

FIG. 40 is a review of the locking screw system depicted within FIG. 39 in place in the pelvic girdle and stabilizing the sacrum.

FIG. 41 is a rear view of a locking screw system such as depicted within FIG. 39, in tandem, in place in the pelvic girdle and stabilizing the sacrum.

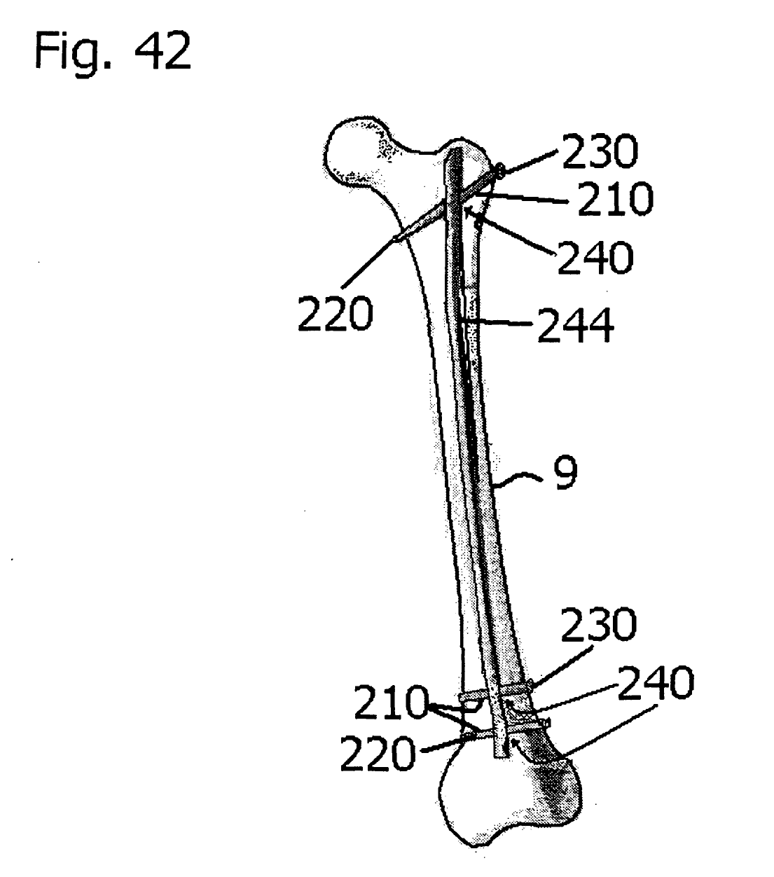

FIG. 42 is a plan view of a locking screw system embodied as a femoral nail, which has major-major and/or minor-minor diameter interference in a plurality of female threaded receiver sites for a plurality of screws, in place in a femur.

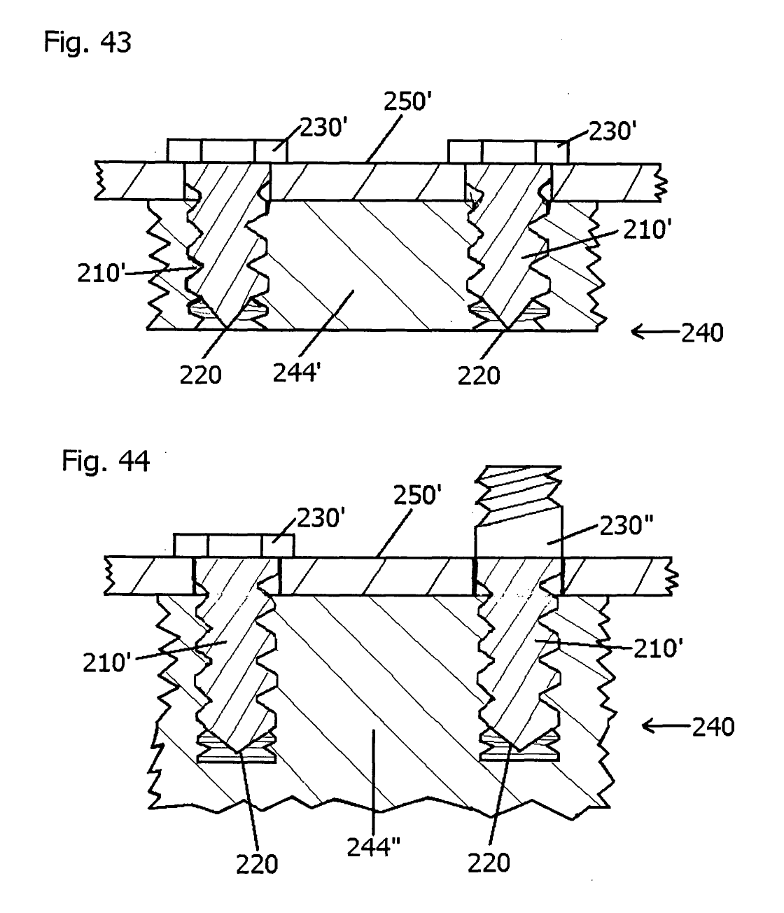

FIG. 43 is a side view in section of a locking screw system in a mechanical system, which has major-major diameter interference in a plurality of open-holed female threaded receiver sites for a plurality of screws, securing a member that may be considered to correspond to a washer.

FIG. 44 is a side view in section of a locking screw system in a mechanical system, which has major-major diameter interference in a plurality of blind-holed female threaded receiver sites for a plurality of screws, securing a member that may be considered to correspond to a washer.

The invention can be further understood by the detail set forth below, which may be read in view of the drawings. As with the foregoing, the following is to be taken in an illustrative and not necessarily limiting sense.

The present implant system includes, in combination, a screw that locks to another part of the system through an interference fit. The interference fit may be through major-minor pitch interference with male threads of the screw and female threads of a nut as a part or a female-threaded portion of the part, for example, a female-threaded portion of a nail or plate; through fitting of deformable threads of a screw into a smooth orifice generally that is harder than the deformable threads, where the deformable thread diameter is greater than an effective distance across the orifice through which the screw passes and deforms the threads, i.e., an oversize thread situation; and/or through other means, say, a harder thread engaging a softer surface of an orifice, and so forth. Any suitable pitch and diameter of threads may be employed. The screw and/or other part may come themselves from prior art provisions, or the screw and/or other part may be provided as novel item(s).

In addition to or in lieu of major-minor pitch diameter interference, the instant interference fit can be through major-major or minor-minor diameter interference. Both major-major and minor-minor diameter interference may be present in the same screw and female threaded receiver system; both may be present independently in one system as a first screw and female threaded receiver system with major-major diameter interference, and a second screw and female threaded receiver with minor-minor diameter interference spaced apart from but fastening the same member as the first; both may be present with at least one of such first and second screw and threaded female receiver systems, plus a third screw and female threaded receiver system having both major-major and minor-minor diameter interference; and so forth.

In the major-major diameter interference, the major outside diameter of the screw threads, i.e., male threads, is larger than the major inside diameter of the female threaded receiver. The male threads of the screw can be in a configuration such as where they are formed with a thread die to leave a peak as a crest that defines the major outside diameter, and the female threads can be in a form of a flattened or “filled in” trough that defines the major inside diameter of the female threaded receiver. A lead-in tip on the screw can be provided, which can have one or a few male threads that have an major outside diameter less than the major inside diameter but larger than the minor insider diameter of the female threaded receiver. When screwed in, the male thread crests may be crushed by the female flattened trough to provide the interference. A follow up chance may be afforded with major-major diameter interference should a preceding screw in the major-major system have a body of a less than desirable size, say, length, or configuration; for example a first screw may be found to have insufficient length to obtain purchase on a section of cortical bone following its passage through a corresponding female threaded receiver in a form of a femoral or tibial nail, be withdrawn from the female threaded receiver by unscrewing, and be replaced by a second screw with a more suitable length.

In minor-minor diameter interference, the minor outside diameter of the screw threads, i.e., the male threads, is larger than the minor inside diameter of the female threaded receiver. The male threads of the screw can be in a form of a flattened or “filled in” trough that defines the minor outside diameter of the male screw threads, and the female threads can be in a configuration such as where they are formed with a thread tap to leave a peak as a crest that defines the minor inside diameter of the female threaded receiver. A lead-in tip on the screw can be provided, which can have one or a few male threads that have a major outside diameter less than the major inside diameter and larger than the minor inside diameter of the female threaded receiver, and that have a minor inside diameter less than the minor inside diameter of the minor inside diameter of the female threaded receiver. When screwed in, the female crests may be crushed by the male flattened trough to provide the interference. A follow up chance may be afforded with minor-minor diameter interference should a preceding female threaded receiver in the minor-minor system have a body of a less than desirable size or configuration; for example, a first nut for an orthopedic implant may be found to not secure a fracture sufficiently, be unscrewed from the corresponding screw, and replaced with a second nut having a more suitable body configuration for the fracture.

The major-major and/or minor-minor diameter interference may be considered a self-locking thread form based on a twist-locking principle. It can be adapted to any screw diameter, thread form or thread pitch. It works with all implant grade material, stainless steel, titanium and its alloys, and cobalt and its alloys, as well as other grade and type materials. One big advantage of this self-locking thread form based on a twist-locking principle is that it provides control of torque to fit the product needs. The torque control is provided through the materials employed to make the male screw and female receiver, the pitch of the threads, as well as predetermined differences in major-major and/or minor-minor diameters. For example, with a major-major diameter interference system made with implant grade stainless steel, titanium or an alloy thereof and/or cobalt or an alloy thereof, male thread crests may define a 0.250-inch (0.984-cm) major outside diameter to the screw with female flattened troughs defining a 0.249-inch (0.980-cm) major inside diameter to the female receiver so as to provide an implant system that can be turned by hand by a surgeon for locking in the operating room. With the same material and pitch between threads, an increase in the difference between the diameters would increase torque, and a decrease between the diameters would decrease torque.

With reference to the drawings, the following is noted:

The screw, for instance, of suitable biologically compatible metal, may be cannulated screw 10 or non-cannulated screw 10′, 10″ and have hollow body 11 or solid body 11′, 11″. The body 11, 11′, 11″ extends along central axis 15. Threads of the screw may be of the bone screw type, say, cancelleous bone screw threads 16; or may be of the deformable type, say, crushable threads 16′, 16″ that are larger in diameter than the effective distance across the orifice of harder material, which may be flattened 16F after passing through the orifice. The screw 10 has tip 20, which can have flute(s) 21 and tapping tongue(s) 23. The screws 10′, 10″ have pointed tip 20′, 20″, respectively. The screws 10, 10′, 10″ each respectively have head 30, 30′, 30″ which respectively have tool-engaging artifice 31, 31′, 31″.

Nut 40, for instance, of suitable biologically compatible metal or of plastic, say, polyacetal resin, may comprise the other part of the system. The nut 40 has suitable tool-engaging artifice 41, and may have shoulder 42 for resting on the bone. The nut 40 may be of the locknut variety when employed in conjunction with a screw, say, the screw 10, and has throughbore 43 in body 44, and internal threads defined by a helical crest and trough arrangement corresponding to the threads 16 of the screw 10 for threading on the screw 10. The threads 46 can be of a bone screw type and be generally correspondent with the threads 16 of the screw 10, for example, also being of the cancellous type. For the interference fit, screw entry internal dimension 48, say, at the trough by the shoulder 42 end of the throughbore 43, is slightly greater than the screw exit internal dimension 49, say again, at the trough but by the opposing end of the throughbore 43, for instance, providing for an about 0.010-inch (ca. 0.25-mm) difference between troughs at the screw entry end versus the screw exit end of the nut 40. The threads 46 may have crests similarly tapered throughout the nut bore 43 so as to provide for an interference fit difference along with the difference provide from the trough dimensions 48, 49, which provides for the screw 10 to nut 40 major-minor, pitch-diameter locking contact, for example, direct metal-to-metal fastening and locking contact, which engenders significantly less risk of loosening. Either the crest or the trough features themselves may provide for the interference fit. Such features of the nut 40 may be provided to a nail, plate, tray, shell or other part of the implant system for interference fitting with the screw 10, 10′, 10″ and the thus outfitted nail, plate, tray, shell or other part.

Washer(s), for instance, of suitable biologically compatible metal or of plastic, say, polyacetal resin, may be provided such as washer 50 and/or washer 50′. These respectively have a bone interfacing surface that is flat 51 or curved 51′ and head engaging cup 52, 52′ with a beveled or concave shape in throughbore 53, 53′.

Nail 60, for instance, of suitable biologically compatible metal has body with first end 61, and second end 62 that has blind lengthwise orifice 63. Transverse orifices 63′, 63″ go through the body, with the oval orifices 63′ passing through the body and having smooth walls the effective distance 63D; and with the round orifices 63″ passing through the body and across the path of the lengthwise orifice 63 and having smooth walls on one end and threads 63T on the other. The threads 63T may conform to the descriptions and depictions of the threads 46 in the nut 40. The nail 60 may be employed with a suitable screw, for example, the screw(s) 10, 10′.

A plate, for instance, of suitable biologically compatible metal, may be employed with a suitable screw, for example, the screw(s) 10, 10′. Flat plate 70 is a broad plate, for example, which, for example, may be employed on the femur or humerous, and it includes beveled, contoured orifices 73. Plate 70′ is a more narrow plate, and it includes beveled, contoured orifices 73′. Special plate 80 is a spoon plate for epiphyseal and/or metaphyseal areas near joints, and it includes beveled, contoured orifices 73. Ligament fixation plate 90 includes orifice 93. Screw(s), for example, the screw 10 and/or the screw 10′ may be passed with interference fitting through the orifice(s) 73, 73′, 83 or 93.

Spiked washer 90W for ligament fixation, for instance, of polyacetal resin with stainless steel for X-ray contrast, may be employed with the screw 10, 10′ or any other screw suitable for practice of the present invention. A screw such as the screw 10 and/or the screw 10′ may be passed with interference fitting through orifice 93W.

Femoral nail 100, for instance, of suitable biologically compatible metal, includes elongate, curved body 101 with throughbore 102 and transverse orifices 103, 103′. A suitable screw such as the screw 10 and/or the screw 10′ may be passed with interference fitting through orifice(s) 103, 103′.

Tibial tray 110, for instance, of suitable biologically compatible metal, includes tray body 111 having orifices 113 through the floor of the tray body 111. Walls of the orifices 113 may be provided with threads 113T through one or more of which a suitable screw, for example, the flat head screw 10″, may be passed with interference fitting therethrough. Distal tibial spike 114 may be provided on the underside of the tray body 111, and porous coating 116 for ingrowth of bone may be provided on the underside of the tray 111 and/or on suitable portion(s) of the distal tibial spike 114.

Acetabular shell 120, for instance, of suitable biologically compatible metal, includes shell body 121 having orifices 123 through a wall of the shell 121. Walls of the orifices 123 may be provided with threads 123T through one or more of which a suitable screw, for example, screw(s) such as the flat head screw 10″, may be passed with interference fitting therethrough. Coxcomb spike 124 may be provided on the body 121, and porous coating 126 for ingrowth of bone may be provided on the outside of the shell body 121.

Glenoid shell 130, for instance, of suitable biologically compatible metal, includes shell body 131 having orifices 133 through a wall of the shell 121. Although walls of the orifices 133 may be provided with threads, the walls of the orifices may be smooth, through one or more of which walls a suitable screw, for example, screw(s) such as the flat head screw 10″, may be passed with interference fitting therethrough. Porous coating 136 for ingrowth of bone may be provided on the outside of the shell body 131.

In addition to or in lieu of the foregoing, major-major and/or minor-minor diameter interference can be provided.

As an orthopedic implant, the locking screw system can be implanted in suitable bone stock 9. The bone stock 9 may be appropriately prepared beforehand by the surgeon.

Centered along the axis 15 are the threaded shaft of male threaded screw 210 and threaded throughput of female threaded receiver 240. The screw 210 can include body 211/210′, which may be solid or have cannulation 215, and may include crests 216 to its threads, which may typically be flat surfaces substantially or exactly parallel to the axis 15, and of diameter 216D, which in major-major diameter interference may be crushed to have crushed crest 216C upon threading into an appropriately corresponding female threaded receiver 240. The female threaded receiver has body 244/244″244″, crushing surfaces 246 substantially or exactly parallel to the axis 15 and of diameter 246D, which is smaller than the diameter 216D, and typical flat crest truncation 247 that helps provide for performance enhancing minor diameter clearance 270. Pitch diameter clearance 260, which would work with any class of thread, may further enhance performance. The screw 210 can include tip 220, which may lead into lead-in threads 223, which are smaller than the diameter 246D, thus not being a cause for interference with the surface 246 and save forward threads from interference in case a screw needs to backed out, and which, in case of a locking screw system for some orthopedic applications, may be provided with bone cutting flutes. The screw 210 accordingly may be self-piloting and self-tapping. The screw 210 has head 230/230′. In minor-minor diameter interference, the screw 210 can have thread troughs 216T with diameter 216D′, which are substantially if not exactly parallel to the axis 15. The female threaded receiver 240 may have threads with inwardly directed crests 247P, which may typically be flat surfaces, along diameter 247D. When screwed in, the crests 247P may be crushed by the troughs 216T to form crushed crest 247C, which provide interference. Washer 250, 250′, 250″ may be employed as a spacer or as a plate or other mechanical part, fastened implant component part, and so forth and the like.

A screw otherwise such as the screw 10, 10′, 10″ may be provided with male threads suitable for major-major and/or minor-minor diameter interference; and a nut otherwise such as the nut 40 may be provided with female threads suitable for major-major and/or minor-minor diameter interference. Among other examples as well, a nail otherwise such as the nail 60, a plate such as the plate 70, 70′, 80 and 90, a spiked washer such as the spiked washer 90W, a femoral nail otherwise such as the femoral nail 100, a tibial tray such as the tibial tray 110, an acetabular shell such as the acetabular shell 120, and a glenoid shell such as the glenoid shell 130 may be provided with threads suitable for major-major and/or minor-minor diameter interference, for instance, with suitable female threads.

Accordingly, an implant or implant component can be provided. The same may be embodied for orthopedic surgery.

Also, among other things, a stud, for example, stud 230″, may be provided with at least one end having threads suitable for major-major and/or minor-minor diameter interference. In addition, numerous other things can be provided with major-major and/or minor-minor diameter interference in accordance with the present principles.

A kit may be provided. For example, the kit may contain one or more of the screw 210 or screw such as the screw 10, 10′, 10″ having male threads suitable for major-major and/or minor-minor diameter interference; one or more of the female threaded receiver 240 or other female threaded receiver such as the nut 40, nail 60, which may be a tibial, humeral or ulnar nail, plate 70, 70′, 80, 90, spiked washer 90W, femoral nail 100, tibial tray 110, acetabular shell 120 or glenoid shell 130; optionally one or more of a washer or other component such as the washer 50, 50′, 250, 250′, 250″; and so forth. Tool(s) may be provided separate from or with the foregoing. The tool(s) are adapted for installation of the locking screw system. For example, the tools may be for helping implant a transiliac locking screw system, which may include guidewire 360 that may be rigid and have drill bit tip 361; cannulated starter drill 370 with bit 371 and bore 372; additional tool set 380, which may include cannulated T-handle Allen wrench 383 and cannulated T-handle socket wrench 384. Compare, Randall et al., U.S. Pat. No. 6,635,059 B2.

The present invention is thus provided. Various feature(s), part(s), subcombination(s) and/or combination(s) may be employed with or without reference to other feature(s), part(s), subcombination(s) and/or combination(s) in the practice of the invention, and numerous and sundry adaptations and modifications can be effected within its spirit, the literal claim scope of which is particularly pointed out as follows:

1. A locking implant system, comprising:
a structural surgical repair implant having an orifice and positioned to stabilize a bone structure;
an internal threaded female receiver within said orifice having internal threads where said internal threads have an internal major diameter and an internal minor diameter; and
an externally threaded male fastener having external threads where said external threads have an external major diameter and an external minor diameter and where said external major diameter is sufficiently greater than the internal major diameter and where said fastener is screwed through said female receiver to thereby deform a crest of the external thread thereby mounting and locking the surgical repair implant.

2. The system of claim 1, where the bone structure is a bone structure selected from the group of bone structures consisting of a tibial, humeral or ulnar nail, a femoral nail, a plate, a spiked washer, a tibial tray, a femoral component for a knee implant, an acetabular shell, and a glenoid shell.

3. The locking implant system of claim 1, where the external minor diameter is sufficiently greater than the internal minor diameter such that the a trough of the external thread is deformed.

4. (canceled)

5. The system of claim 1, wherein the the externally threaded male fastener includes threads with crests that have an external major diameter; and the internal threaded female receiver includes threads with crushing surfaces substantially if not exactly parallel to an axis central to the fastener and receiver and having an internal major diameter, which is smaller than an external major diameter of the fastener.

6. The system of claim 5, wherein the fastener is a screw having a tip adapted for piloting through the internal threaded female receiver; and the internal threaded female receiver has inwardly facing crests that are truncated.

7. The system of claim 6, which is embodied as or with an implant or implant component for orthopedic surgery.

8-23. (canceled)

24. A locking implant system, comprising:
a structural surgical repair implant having an orifice and positioned to stabilize a bone structure;
an internal threaded female receiver within said orifice having internal threads where said internal threads have an internal major diameter and an internal minor diameter; and
an externally threaded male fastener having external threads where said external threads have an external major diameter and an external minor diameter and where said external minor diameter is sufficiently greater than the internal minor diameter and where said fastener is screwed through said female receiver to thereby deform a trough of the external thread thereby mounting and locking the surgical repair implant.

25. The system of claim 24, where the bone structure is a bone structure selected from the group of bone structures consisting of a tibial, humeral or ulnar nail, a femoral nail, a plate, a spiked washer, a tibial tray, a femoral component for a knee implant, an acetabular shell, and a glenoid shell.

26. The system of claim 24, wherein the externally threaded male fastener includes threads with troughs that have an external minor diameter; and the internal threaded female receiver includes threads with crushing surfaces substantially if not exactly parallel to an axis central to the fastener and receiver and having an internal minor diameter, which is smaller than an external minor diameter of the fastener.

27. The system of claim 26, wherein the fastener is a screw having a tip adapted for piloting through the internal threaded female receiver; and the internal threaded female receiver has inwardly facing troughs that are truncated.

28. The system of claim 26, which is embodied as or with an implant or implant component for orthopedic surgery.

29. The system of claim 24, where a crest of the internal thread is deformed.
